(19) United States
(12) Patent Application Publication
(10) Pub. No.: US 2023/0225785 A1
(43) Pub. Date: Jul. 20, 2023

(54) COMPOSITE COATING FOR ELECTROSURGICAL ELECTRODE

(71) Applicant: Covidien LP, Mansfield, MA (US)

(72) Inventors: Xinmeng Liu, Shanghai (CN); Tong Shen, Shanghai (CN); Lijun Zhu, Shanghai (CN); Yongming Zhao, Shanghai (CN); Fang Geng, Shanghai (CN); Peng Zha, Shanghai (CN)

(73) Assignee: Covidien LP, Mansfield, MA (US)

(21) Appl. No.: 18/010,232

(22) PCT Filed: Jul. 16, 2020
(86) PCT No.: PCT/CN2020/102269

§ 371 Date: Dec. 14, 2022

(51) Int. Cl. A61B 18/14

(52) U.S. Cl. CPC A61B 18/1402; A61B 2018/00136

(57) ABSTRACT

An electrosurgical electrode (30) includes a conductive rod (32) having a working portion (38) at a distal end portion (35). The electrode (30) also includes a composite coating (40) disposed on the working portion (38). The composite coating (40) includes a first coating (42) disposed on an outer surface of the working portion (38) and a second coating (44) disposed over the first coating (42).

### BACKGROUND

### Technical Field

The present disclosure relates to an electrosurgical electrode and, more particularly, to an electrosurgical electrode including a composite coating.

### Background of Related Art

Electrosurgery involves application of high radio frequency (RF) electrical current to a surgical site to cut, ablate, desiccate, or coagulate tissue. In monopolar electrosurgery, a source or active electrode delivers radio frequency alternating current from the electrosurgical generator to the targeted tissue. A patient return electrode is placed remotely from the active electrode to conduct the current back to the generator.

Monopolar electrodes apply RF electrical energy to heat the tissue to transect or achieve hemostasis. Thus, there is a need for a coating which can reduce the unexpected thermal damage and secondary damage caused by tissue adhesion during application of RF energy.

### SUMMARY

Polytetrafluoroethylene (PTFE) coating disposed directly on electrode surface may decompose and peel from the electrode due to high temperature and arcing during application of RF energy. This may cause a decrease in blade performance due to tissue sticking to the surface of the electrode.

The present disclosure provides an electrosurgical electrode, such as a monopolar blade electrode used in open surgery and laparoscopic surgery, having a composite coating, which is used to prevent secondary damage caused by intraoperative thermal damage and tissue adhesion. The electrode includes a composite coating having a PTFE primer coating and a second coating formed of perfluoroalkoxy alkanes (PFA), which is a copolymer of hexafluoropropylene and perfluoroethers. Compared with traditional PTFE coated monopolar blade, composite coating has better surface adhesion and anti-sticking performance.

According to one embodiment of the present disclosure, an electrosurgical electrode is disclosed. The electrode includes a conductive rod having a working portion at a distal end portion. The electrode also includes a composite coating disposed on the working portion. The composite coating includes a first coating disposed on an outer surface of the working portion and a second coating disposed over the first coating.

According to another embodiment of the present disclosure, an electrosurgical electrode is disclosed. The electrode includes a conductive rod including a distal end portion having a working portion and a proximal end portion configured to couple to an electrosurgical instrument. The electrode also includes a composite coating disposed on the working portion. The composite coating includes a first coating formed from a first polymer disposed on an outer surface of the working portion and a second coating disposed over the first coating, the second coating formed from a second polymer, different from the first polymer.

According to one aspect of any of the above embodiments, the outer surface of the working portion has a roughness from about 0.6 Ra to about 0.8 Ra. The first coating may include polytetrafluoroethylene. The second coating may be a powder coating of perfluoroalkoxy alkanes.

According to another aspect of any of the above embodiments, the first coating has a thickness from about 7 μm to about 9 μm. The second coating has a thickness from 12 μm to about 15 μm. The composite coating has a thickness from about 19 μm to about 24 μm. The second coating has a roughness from about 0.2 Ra to about 0.4 Ra.

According to a further embodiment of the present disclosure, a method for making an electrosurgical electrode is disclosed. The method includes texturing a working portion of an electrosurgical electrode; applying a first coating formed from a first polymer to an outer surface of the working portion; and applying a second coating onto the first coating, the second coating formed from a second polymer, different from the first polymer.

According to one aspect of the above embodiment, texturing including sandblasting the working portion to have a roughness from about 0.6 Ra to about 0.8 Ra. Applying the first coating may also include achieving a thickness from about 7 μm to about 9 μm for the first coating. Applying the second coating may also include achieving a thickness from about 19 μm to about 24 μm for the second coating.

### BRIEF DESCRIPTION OF THE DRAWINGS

The present disclosure may be understood by reference to the accompanying drawings, when considered in conjunction with the subsequent, detailed description, in which:

FIG. 1 is a perspective view of an electrosurgical system according to an embodiment of the present disclosure;

FIG. 2 is a perspective view of an electrode according to an embodiment of the present disclosure;

FIG. 3 is a side cross-section view of the electrode of FIG. 2 taken along a cross-sectional line 2-2;

FIG. 4 is a photograph of a coating of the electrode of FIG. 2 electrode according to an embodiment of the present disclosure;

FIGS. 5-9 are photographs of porcine liver tissue cut with the electrode of FIG. 2, an uncoated electrode, a PTFE coated electrode, and silicone coated electrode; and

FIG. 10 is a table summarizing observations of photographs of FIGS. 5-9.

### DETAILED DESCRIPTION

Embodiments of the presently disclosed electrosurgical system are described in detail with reference to the drawings, in which like reference numerals designate identical or corresponding elements in each of the several views. As used herein the term “distal” refers to the portion of the surgical instrument coupled thereto that is closer to the patient, while the term “proximal” refers to the portion that is farther from the patient.

In the following description, well-known functions or constructions are not described in detail to avoid obscuring the present disclosure in unnecessary detail. Those skilled in the art will understand that the present disclosure may be adapted for use with either an endoscopic instrument, a laparoscopic instrument, or an open instrument. It should also be appreciated that different electrical and mechanical connections and other considerations may apply to each particular type of instrument.

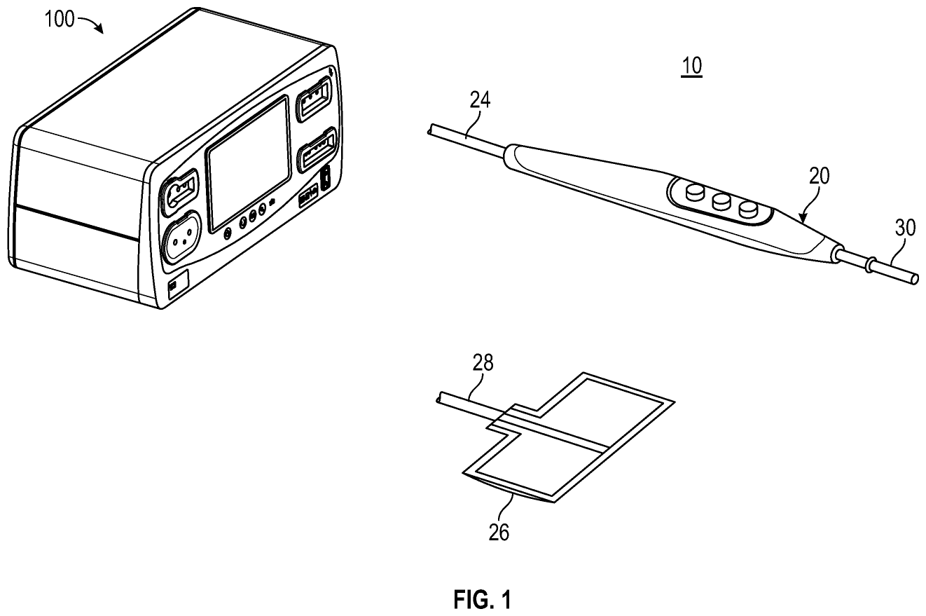

Referring to FIG. 1 an electrosurgical system 10 for use with an electrosurgical instrument having an electrode according to the present disclosure, such as a monopolar electrosurgical instrument 20. Monopolar electrosurgical instrument 20 includes an active electrode 30 (e.g., electrosurgical cutting blade, etc.) for treating tissue of a patient. The system 10 may include a plurality of return electrode pads 26 that, in use, are disposed on a patient to minimize the chances of tissue damage by maximizing the overall contact area with the patient. Electrosurgical alternating RF current is supplied to the monopolar electrosurgical instrument 20 by a generator 100 via supply line 24. The alternating RF current is returned to the generator 100 through the return electrode pad 26 via a return line 28.

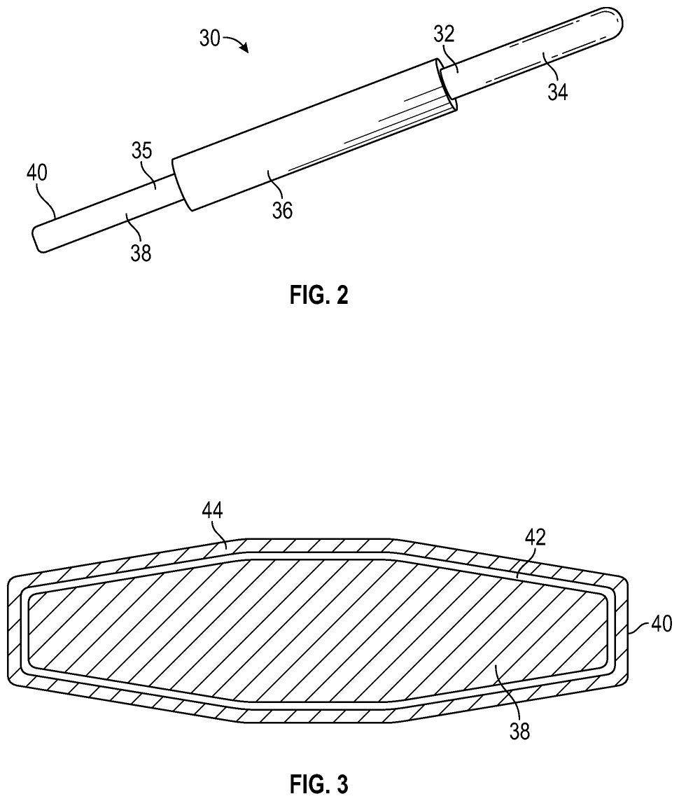

With reference to FIG. 2, the electrode 30 is formed from a conductive type material, such as, stainless steel. The electrode 30 may be shaped as a longitudinal rod 32 having a proximal end 34 configured to couple to the instrument 20. The electrode 30 has an insulative portion 36, which may be a coating or an insulative sleeve disposed over a middle portion of the longitudinal rod 32 leaving the proximal end portion 34 and a distal end portion 35 exposed. The electrode 30 also includes a working portion 38 at its distal end portion 35. The working portion 38 may be shaped like a blade or any other suitable structure, such as a spatula, a hook, a needle, etc.

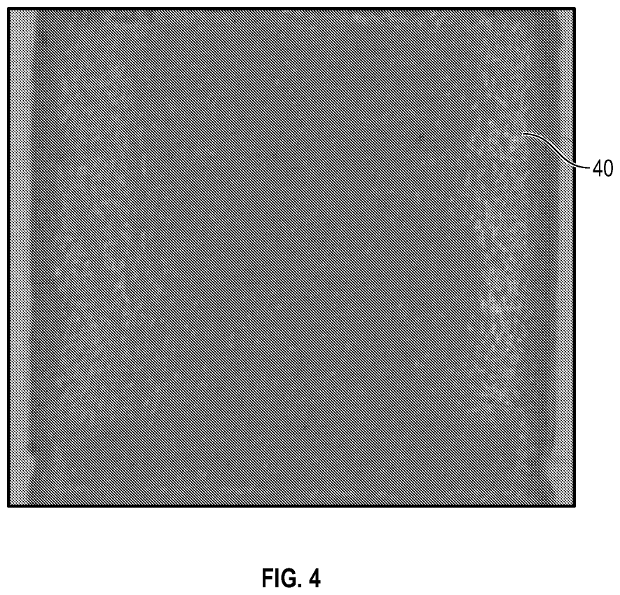

The working portion 38 includes a composite coating 40 disposed on its outer surface. With reference to FIG. 3, the cross-sectional view of the working portion 38 with the composite coating 40 having a first (e.g., bottom, inner) coating 42 and a second (e.g., top, outer) coating 44. The working portion 38 has a rough texture to provide for better adherence of the first coating 42. The roughened texture may be achieved by sandblasting or any other suitable technique, such as, etching, of the working portion 38. The surface of the working portion 38 may have a roughness from about 0.6 Ra to about 0.8 Ra.

After the working portion 38 is roughened, the first coating 42 is applied to achieve a desired thickness. The first coating 42 may have a thickness from about 7 μm to about 9 μm. The first coating 42 is formed from a polymer, such as PTFE, which may be applied by atomizing or aerosolizing a PTFE solution using a high-pressure air supply and spraying the PTFE solution on the surface of the working portion 38. Thereafter, the first coating 42 is dried and sintered.

Once the first coating 42 has solidified, the second coating 44 is applied to the first coating 42. The second coating 44 may be formed from a second polymer, that is different from the first polymer of the first coating 42. The second coating 44 may be a powder coating formed from PFA particles and may be formed by spraying onto the first coating 42 until a desired thickness is achieved. The second coating 44 may have a thickness from about 12 μm to about 15 μm. The composite coating 40 may have a combined thickness from about 19 μm to about 24 μm.

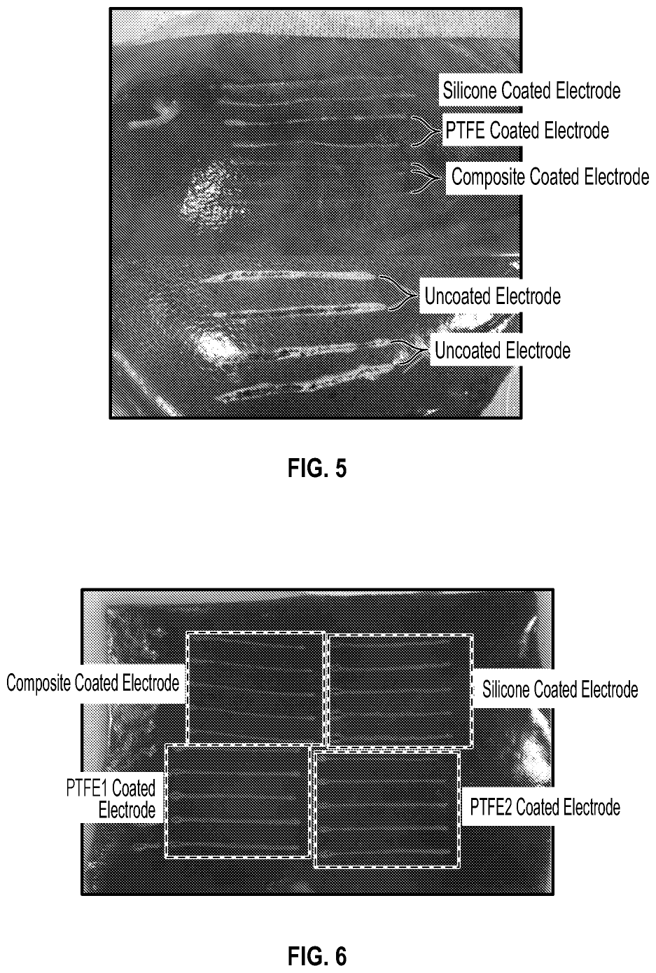

With reference to FIG. 4, an enlarged photograph of the coating 40 is shown illustrating the surface roughness of the coating 40 and its uniformity. Roughness of the second coating 44 may be from about 0.2 Ra to about 0.4 Ra. Thus, the coating 40 is smoother than the substrate of the working portion 38. The relatively thin thickness of the dual-layer coating 40 allows for desired electrical performance of the electrode 30 while providing tissue sticking reduction. In addition, the electrode 30 having the coating 40 may be used continuously at a temperature from about 260° C. to about 290° C.

The following Examples illustrate embodiments of the present disclosure. These Examples are intended to be illustrative only and are not intended to limit the scope of the present disclosure. Also, parts and percentages are by weight unless otherwise indicated. As used herein, “room temperature” or “ambient temperature” refers to a temperature from about 20° C. to about 25° C.

### EXAMPLES

### Example 1

This Example describes effectiveness of the dual-layer PTFE/PFA coating according to the present disclosure as compared to uncoated, silicone, and single layer PTFE coated electrodes.

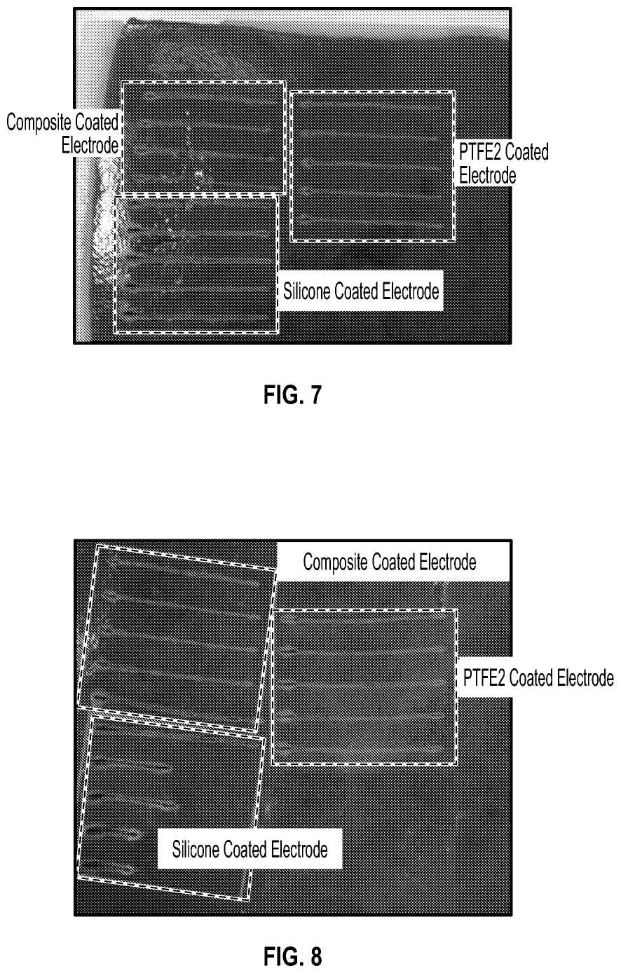

Four electrodes were used to determine effectiveness of the coating of the present disclosure including an uncoated electrode, a silicone coated electrode, a PTFE coated electrode, and a composite coated electrode according to the present disclosure. Each of the electrodes were used with a VALLEYLAB™ FT10® generator available from Medtronic of Minneapolis, Minn. in a manual cut mode at 10 Watts setting. The electrodes were used to make incisions in porcine liver tissue and are shown in FIG. 5. The blade cutting marks of electrode having the composite coating were narrower compared with cuts made by other electrodes. Also, thermal spread of cut performance was smaller than that of other electrodes.

A fatigue test was also performed on the four electrodes to determine their effectiveness after multiple cuts. The uncoated electrode was substituted with another PTFE coated electrode (PTFE 2). For the fatigue test, twenty cuts were made with each electrode and the electrodes were tested until failure of the coatings to evaluate durability of coatings. The electrodes were mounted to a Gantry system to control cutting length, depth, and speed. In particular, the electrodes were used to make a 40 mm cut, having approximately a 2 mm depth, at a speed of about 10 mm/s. During cutting the generator was in manual cutting mode at 15 Watts setting. FIGS. 6-9 illustrate cuts made in porcine liver tissue by each of the electrodes in groups of five cuts. Thus, each of the FIGS. 6-9 shows four rounds of five cuts each.

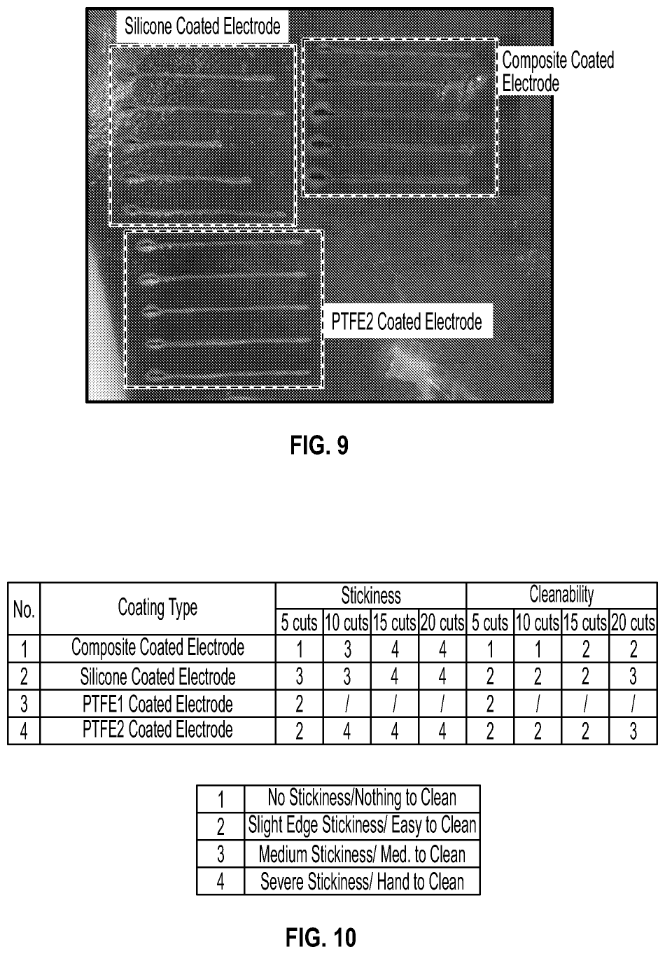

The first 1-15 cuts, width of cuts made with the composite coated electrode were narrower than those made with electrodes having other coatings. Furthermore, the first PTFE coated electrode (PTFE1) failed after 10 cuts. After 20 cuts, the silicone coated electrode failed to cut completely and could not form unbroken cut marks whereas the composite coated electrode cut smoothly and flatly. In addition, stickiness and cleanability of each of the electrodes was evaluated and the results are included in the table of FIG. 10. The composite coated electrode also outperformed the other three coated electrodes.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope of the claims appended hereto.

What is claimed is:

1. An electrosurgical electrode comprising:
a conductive rod having a working portion at a distal end portion;
a composite coating disposed on the working portion, the composite coating including a first coating disposed on an outer surface of the working portion and a second coating disposed over the first coating.

2. The electrosurgical electrode according to claim 1, wherein the outer surface of the working portion has a roughness from about 0.6 Ra to about 0.8 Ra.

3. The electrosurgical electrode according to claim 1, wherein the first coating includes polytetrafluoroethylene.

4. The electrosurgical electrode according to claim 1, wherein the second coating is a powder coating of perfluoroalkoxy alkanes.

5. The electrosurgical electrode according to claim 1, wherein the first coating has a thickness from about 7 μm to about 9 μm.

6. The electrosurgical electrode according to claim 1, wherein the second coating has a thickness from 12 μm to about 15 μm.

7. The electrosurgical electrode according to claim 1, wherein the composite coating has a thickness from about 19 μm to about 24 μm.

8. The electrosurgical electrode according to claim 1, wherein the second coating has a roughness from about 0.2 Ra to about 0.4 Ra.

9. An electrosurgical electrode comprising:
a conductive rod including a distal end portion having a working portion and a proximal end portion configured to couple to an electrosurgical instrument;
a composite coating disposed on the working portion, the composite coating including a first coating formed from a first polymer disposed on an outer surface of the working portion and a second coating disposed over the first coating, the second coating formed from a second polymer, different from the first polymer.

10. The electrosurgical electrode according to claim 9, wherein the outer surface of the working portion has a roughness from about 0.6 Ra to about 0.8 Ra.

11. The electrosurgical electrode according to claim 9, wherein the first polymer is polytetrafluoroethylene.

12. The electrosurgical electrode according to claim 9, wherein the second polymer includes perfluoroalkoxy alkanes.

13. The electrosurgical electrode according to claim 9, wherein the first coating has a thickness from about 7 μm to about 9 μm.

14. The electrosurgical electrode according to claim 9, wherein the second coating has a thickness from 12 μm to about 15 μm.

15. The electrosurgical electrode according to claim 9, wherein the composite coating has a thickness from about 19 μm to about 24 μm.

16. The electrosurgical electrode according to claim 9, wherein the second coating has a roughness from about 0.2 Ra to about 0.4 Ra.

17. A method for making an electrosurgical electrode, the method comprising:
texturing a working portion of an electrosurgical electrode;
applying a first coating formed from a first polymer to an outer surface of the working portion; and
applying a second coating onto the first coating, the second coating formed from a second polymer, different from the first polymer.

18. The method according to claim 17, wherein texturing including sandblasting the working portion to have a roughness from about 0.6 Ra to about 0.8 Ra.

19. The method according to claim 17, wherein applying the first coating includes achieving a thickness from about 7 μm to about 9 μm for the first coating.

20. The method according to claim 17, wherein applying the second coating includes achieving a thickness from about 19 μm to about 24 μm for the second coating.
